(19) United States
(12) Patent Application Publication
(10) Pub. No.: US 2016/0008375 A1
(43) Pub. Date: Jan. 14, 2016

(54) PHARMACEUTICAL FORMULATIONS COMPRISING QUETIAPINE AND ESCITALOPRAM

(71) Applicant: SANOVEL ILAC SANAYI VE TICARET A.S., Istanbul (TR)

(72) Inventors: Ali Turkyilmaz, Istanbul (TR); Müge Ulusoy Bozyel, Istanbul (TR); Yelda Erdem, Istanbul (TR); Isin Ruiye Okyar, Istanbul (TR)

(21) Appl. No.: 14/771,692

(22) PCT Filed: Feb. 27, 2014
(86) PCT No.: PCT/EP2014/053812

§ 371 Date: Aug. 31, 2015

(30) Foreign Application Priority Data
Mar. 1, 2013 (TR) 2013/02478

(51) Int. Cl. A61K 31/554; A61K 9/20; A61K 31/194; A61K 31/343; A61K 9/24

(52) U.S. Cl. CPC A61K 31/554; A61K 31/343; A61K 9/209; A61K 31/194; A61K 9/2059; A61K 9/2054; A61K 9/2009; A61K 9/2027; A61K 9/2013

(57) ABSTRACT

The present invention relates to a multilayer tablet formulation comprising a combination of quetiapine or a pharmaceutically acceptable salt thereof and escitalopram or a pharmaceutically acceptable salt thereof.

### FIELD OF INVENTION

The present invention relates to a formulation comprising a combination of quetiapine or a pharmaceutically acceptable salt thereof and escitalopram or a pharmaceutically acceptable salt thereof. The present invention more particularly relates to a multilayer tablet formulation comprising quetiapine and escitalopram.

### BACKGROUND OF INVENTION

Quetiapine fumarate, a dibenzothiazepine derivative, is an atypical antipsychotic. It ameliorates the negative and positive symptoms of schizophrenia, without giving rise to extrapyramidal side effects. Similar to clozapine, quetiapine is a moderate dopamine D2-receptor antagonist and a potent selective serotonin reuptake inhibitor. The chemical name of quetiapine fumarate is 2-[2-(4-dibenzo[b,f][1,4]thiazepine-11-yl-1-piperazinyl)ethoxy]ethanol fumarate, with the following chemical structure of Formula-I.

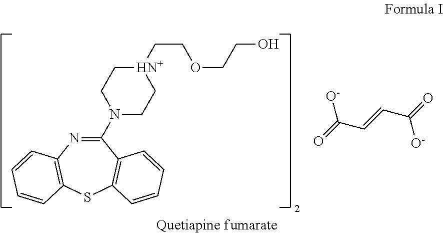

Quetiapine is marketed under the name Seroquel XR® and is administered orally once a day. A unit formulation thereof comprises 50 mg, 150 mg, 200 mg, 300 mg or 400 mg quetiapine fumarate as an active agent.

The quetiapine molecule was first disclosed in the patents EP0240228B1 and U.S. Pat. No. 4,879,288A. The psychotic and hyperactivity indications of quetiapine were also disclosed in these patent documents.

Escitalopram, in turn, is a selective serotonin reuptake inhibitor. Escitalopram is an S-enantiomer of the citalopram molecule and the most frequently used salt of escitalopram in pharmaceutical formulations is escitalopram oxalate. The chemical name of escitalopram is (S)-1-[3-(dimethylamino)propyl]-1-(4-fluorophenyl)1-,3-dihydroisobenzofuran-5-carbonitrile with the following chemical structure of Formula II.

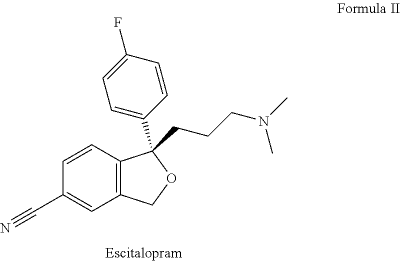 [SMILES: CN(C)CCC[C@@]1(c2ccc(F)cc2)OCc2cc(C#N)ccc21]

The tablet formulations of escitalopram are marketed under the name Cipralex®. Such a unit tablet formulation comprises 10 mg or 20 mg escitalopram oxalate.

Citalopram was first disclosed in the patent U.S. Pat. No. RE34712 of the firm Lundbeck. Escitalopram is used in the treatment of major depressive conditions, panic disorders with agoraphobia or without agoraphobia, social anxiety disorder (social phobia), generalized anxiety disorder and obsessive compulsive disorder (OCD).

The use of more than one drug in the treatment of antidepression has been observed to accelerate the treatment according to the prior art. The use of a combination of fluoxetine, one of the selective serotonin reuptake inhibitors, together with olanzapine, one of the atypical psychotics, in the treatment of psychos, acute mania, mild anxiety conditions, bipolar disorder or depression was disclosed in the patent EP0830864B1. Furthermore, a combination of quetiapine with citalopram in the treatment of OCD was proved to be more effective than citalopram alone as reported in the scientific literature.

So far, no multilayer tablet formulation comprising a combination of quetiapine and escitalopram has been made. Even if selective serotonin reuptake inhibitors and atypical psychotics have been used together in practice, this fact requires the patients to carry more than one drug and gives rise to application-related difficulties.

Despite the fact that at least an additive therapeutic effect is expected for the drugs used in the same therapeutic field or even for the treatment of the same indication, it is well known that these drugs cannot a priori be combined in all cases. The scientific literature is full of examples showing that the compounds from different classes used for treating the same indications cannot always be combined in reliable and efficient dosage forms and therefore may result in incompatible drug combinations. The causes of this unexpected incompatibility are diverse; the typically observed outcomes, however, include the increases in the side effects of different drug combinations, undesired drug interactions, and formation of new side effects.

The basic difficulties encountered when two or more molecules are combined in the same pharmaceutical dosage form are (a) providing the compatibility between different active agents and/or among the active agents and the excipients used, (b) providing therapeutic compatibility between the active agents, taking into account the pharmacokinetic and/or biopharmaceutical properties such as the posology of the respective combination to obtain efficient and reliable plasma levels of both active agents.

Accordingly, there is a need for an immediate-release multilayer tablet formulation comprising quetiapine and escitalopram, which has the aforesaid efficient plasma concentration, is stable, has lower side effects and a high bioavailability.

### DESCRIPTION OF FIGURES

FIG. 1 illustrates an oral solid multilayer tablet formulation having i) a first layer (a) comprising a first active agent, ii) a second layer (b) comprising a second active agent, iii) a inert layer (c) separating these two layers from each other.

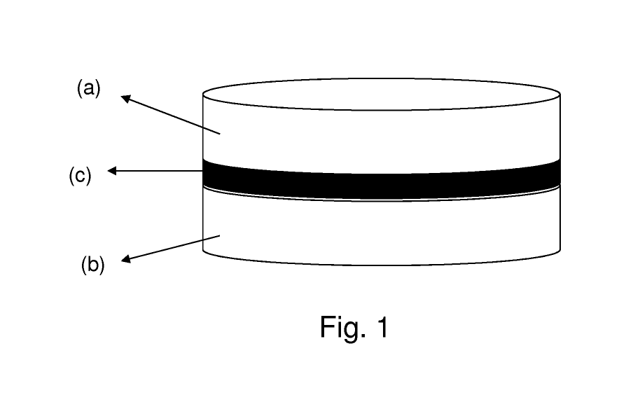

### DETAILED DESCRIPTION OF INVENTION

The present invention relates to an easily-administrable multilayer tablet formulation comprising quetiapine and escitalopram, eliminating all of the aforesaid problems and brining additional advantages to the relevant prior art.

Accordingly, the main object of the present invention is to obtain a stable immediate-release multilayer tablet formulation comprising a combination of quetiapine and escitalopram.

Another object of the present invention is to obtain an immediate-release multilayer tablet formulation having a desired level of dissolution rate by virtue of using suitable excipients. Various formulations are available, which have been developed using quetiapine fumarate. On the other hand, various problems are encountered which are associated with quetiapine fumarate formulations. Quetiapine fumarate has a low dissolution rate. Obtaining a desired release profile depends on the suitability of excipients to be selected. Particularly since the quetiapine molecule is poorly dissolvable, the disintegrant ratio of the layers comprising quetiapine has to be regulated well. Additionally, achieving desired solubility profiles is difficult either, i.e. it is difficult to have both layers to show the same release rate.

Due to the reasons stated above, there is a need in the prior art for a stable pharmaceutical formulation with a high bioavailability and a desired release profile, which comprises a combination of quetiapine or a pharmaceutically acceptable salt thereof and escitalopram or a pharmaceutically acceptable salt thereof.

Thus, the present invention provides a multilayer tablet formulation comprising quetiapine and escitalopram, eliminating all of the aforesaid problems and brining additional advantages to the relevant prior art.

Accordingly, the main object of the present invention is to obtain a stable multilayer formulation with a desired release profile comprising quetiapine and escitalopram.

Another object of the present invention, in turn, is to obtain a multilayer tablet formulation, comprising layers including different active agents and having a desired level of dissolution rate by virtue of employing suitable excipients.

A further object of the present invention is to increase the bioavailability of the tablet formulation by providing a desired level of dissolution rate by virtue of the active agents and the excipients used in the formulation.

In order to achieve all objects referred to above and to be described in the following description, a multilayer tablet formulation is developed which comprises quetiapine or a pharmaceutically acceptable salt thereof and escitalopram or a pharmaceutically acceptable salt thereof. This formulation comprises a layer (a) comprising quetiapine, a layer (b) comprising escitalopram, and an inert layer (c) separating these layers from each other.

In an embodiment according to the present invention, said novelty is embodied by setting the ratio of the amount of disintegrant in the layer comprising quetiapine to the amount of disintegrant in the layer comprising escitalopram between 1/1 and 25/1, preferably between 2/1 and 20/1. According to the present invention, both the layer comprising quetiapine which is poorly-soluble and the layer comprising escitalopram are set to provide immediate release of the active agents at the same time. With the formula according to the present invention, the efficiency of both active agents can be seen at the same times. Additionally, with the formulation according to the present invention, a multilayer tablet formulation can be obtained which has a high bioavailability.

Suitable disintegrants for use in the formulation according to the present invention include, but are not limited to alginic acid and alginates, ion-exchange resins, magnesium aluminum silicate, sodium dodecyl sulfate, sodium carboxymethyl cellulose, croscarmellose sodium, cross linked polyvinylpyrrolidone, carboxymethylcellulose calcium, docusate sodium, guar gum, corn starch, polacrilin potassium, poloxamer, povidone, sodium alginate, sodium glycine carbonate, sodium lauryl sulfate, sodium starch glycolate, or the mixtures thereof.

In a preferred embodiment according to the present invention, it was surprisingly found that two layers each comprising a different active agent are made to provide immediate release at the same times by virtue of setting the ratio of the inert layer thickness to the total tablet thickness between 1/60 and 1/4, preferably between 1/40 and 1/6. Thus, a multilayer tablet formulation is obtained, showing good solubility and therefore having a high bioavailability following oral administration.

In a preferred embodiment according to the present invention, it was further observed that the use of microcrystalline cellulose in different amounts in each layer had an influence on the solubility. The amount of microcrystalline cellulose is 10.0 to 50.0%, preferably 20.0 to 40.0% in the layer comprising quetiapine; 60.0 to 85.0%, preferably 70.0 to 80.0% in the layer comprising escitalopram; 70.0 to 90.0%, preferably 75.0 to 85.0% in the inert layer. The different ratios of microcrystalline cellulose in different layers assist in providing the immediate release of the layers comprising the active agents in the multilayer tablet. Additionally, since microcrystalline cellulose is a stable excipient, it further increased the stability of the tablet according to the present invention.

The formulation according to the present invention may have different unit dosages comprising 400 mg quetiapine and 20 mg escitalopram; 400 mg quetiapine and 10 mg escitalopram; 300 mg quetiapine and 20 mg escitalopram; 300 mg quetiapine and 10 mg escitalopram; 200 mg quetiapine and 20 mg escitalopram; 200 mg quetiapine and 10 mg escitalopram; 100 mg quetiapine and 20 mg escitalopram; 100 mg quetiapine and 10 mg escitalopram.

In a preferred embodiment according to the present invention, quetiapine is used in the form of quetiapine fumarate and escitalopram is used in the form of escitalopram oxalate.

Besides microcrystalline cellulose, the excipients used in the formulation according to the present invention further comprise at least one or a mixture of fillers, binders, lubricants, glidants, and coating agents.

Besides microcrystalline cellulose, the filler used in the formulation according to the present invention is selected from a group comprising mannitol, spray-dried mannitol; dibasic calcium phosphate dihydrate, lactose, sugars, sorbitol, lactose monohydrate; a mixture of mannitol, polyplasdone and syolid; a mixture of mannitol, crospovidone and polyvinyl acetate; isomalt, sucrose, inorganic salts such as calcium salts, or the mixtures thereof.

Suitable binders for use in the formulation according to the present invention is selected from a group comprising natural gums, starch, gelatin, polyvinylpyrrolidone, polymethacrylates; proteins such as gelatin and collagen; agar, alginate, sodium alginate, pectin, starch, carboxymethyl cellulose, hydroxypropyl cellulose, hydroxypropylmethyl cellulose; synthetic polymers such as carbomer, poloxamer, polyacrylamide, polyvinyl alcohol; inorganic substances such as aluminum hydroxide, bentonite, laponite; starch mucilage, acacia mucilage, polydextrose, polyethylene oxide, or the mixtures thereof.

Suitable lubricants or glidants for use in the formulation according to the present invention ais selected from a group comprising sodium stearyl fumarate, magnesium stearate, polyethylene glycol, colloidal silicon dioxide, stearic acid, talk, metal stearates, boric acid, sodium chloride benzoate and acetate, sodium or magnesium lauryl sulfate, or the mixtures thereof.

Suitable coloring agents for use in a formulation according to the present invention include, but are not limited to food, drug, and cosmetic (FD & C) dyes (FD & C blue, FD & C green, FD & C red, FD & C yellow, FD & C lake), ponceau, indigo drug & cosmetic (D & C) blue, indigotine FD & C blue, carmoisine indigotine (indigo Carmine); iron oxides (e.g. iron oxide red, yellow, black), quinoline yellow, flame red, brilliant red (carmine), carmoisine, sunset yellow, or the mixtures thereof.

Suitable coating agents for use in a formulation according to the present invention include, but are not limited to hydroxypropylmethyl cellulose, polyethylene glycol, polyvinylpyrrolidone, polyvinylpyrrolidone-vinyl acetate copolymer (PVP-VA), polymers such as pullulan, and all kinds of Opadry, as well as pigments, Kollicoat IR®, dyes, titanium dioxide and iron oxide, and talk.

The formulation according to the present invention is for use in the treatment and prevention of diseases such as the bipolar disease, obsessive-compulsive disorder and schizophrenia, mania, depression, dementia, panic disorder, social phobia, generalized anxiety disorder, agitation.

### EXAMPLES

Example 1

| | Amount (%) |
|---|---|
| 1^{st} Layer: Quetiapine fumarate | |
| Quetiapine fumarate | 30.00-60.0 |
| Lactose monohydrate | 5.00-40.00 |
| Microcrystalline cellulose | 10.0-50.0 |
| Dibasic calcium phosphate dihydrate | 0.5-20.0 |
| Sodium starch glycolate | 1.0-20.0 |
| Polyvinylpyrrolidone | 0.05-10.0 |
| Magnesium stearate | 0.01-5.0 |
| 2^{nd} Layer: Inert layer: | |
| Hydroxypropyl cellulose | 0.5-15.0 |
| Microcrystalline cellulose PH 102 | 70.00-90.00 |
| Red iron oxide | 0.01-2.0 |
| Colloidal silicon dioxide | 0.01-2.0 |
| Magnesium stearate | 0.01-5.0 |
| 3^{rd} Layer: Escitalopram oxalate | |
| Escitalopram oxalate | 7.0-15.0 |
| Microcrystalline cellulose PH 102 | 60.0-85.0 |
| Talk | 0.1-10.0 |
| Colloidal silicon dioxide | 0.01-2.0 |
| Croscarmellose sodium | 0.1-10.0 |
| Magnesium stearate | 0.01-5.0 |

*The percent amounts given were calculated separately for each layer.

1^{st} Layer:

→ Weighed amounts of quetiapine fumarate (active agent), dibasic calcium hydrogen phosphate dihydrate, microcrystalline cellulose, sodium starch glycolate and lactose monohydrate are charged to a fluidized bed dryer.

→ A granulation process is carried out by spraying a binder solution formed from a mixture of polyvinylpyrrolidone and pure water to the powder fluidized in the fluidized bed dryer.

→ The granules that dried to the desired humidity content in the fluidized bed dryer are passed through a dry mill. Magnesium stearate is added to the mixture and stirred for 3 minutes.

2^{nd} Layer:

→ Weighed amounts of hydroxypropyl cellulose, microcrystalline cellulose PH 102, red iron oxide, colloidal silicon dioxide are charged to a mixer and mixed for 10 minutes.

→ Magnesium stearate is added to the mixture and stirred for 3 minutes.

3^{rd} Layer:

→ Weighed amounts of escitalopram oxalate, microcrystalline cellulose PH 102, half of croscarmellose sodium yellow are taken to a Collette and mixed for 10 minutes.

→ The resulting powder mixture is granulated with 5.00 liters of water in the Collette at a low rpm.

→ Wet granules are taken to a drying oven without sieving.

→ Dried granules are sieved through a 500 micron Frewith sieve together with colloidal silicon dioxide.

→ The remaining half of croscarmellose sodium and talk are added to the sieved granules and mixed for 10 minutes at 12 rpm.

→ Finally, magnesium stearate is added thereto and mixed for 3 minutes at 12 rpm.

The mixtures prepared are compressed into three separate layers and combined in a tablet.

1. A multilayer tablet formulation for oral administration, comprising quetiapine or a pharmaceutically acceptable salt thereof, escitalopram or a pharmaceutically acceptable salt thereof, and one or more excipient.

2. The tablet formulation according to claim 1, comprising a layer comprising quetiapine, a layer comprising escitalopram, and an inert layer separating these two layers from each other.

3. The tablet formulation according to claim 1, wherein the excipient is a disintegrant, and the ratio of the amount of the disintegrant present in the layer comprising quetiapine to the amount of the disintegrant present in the layer comprising escitalopram is between 1/1 and 25/1.

4. The tablet formulation according to claim 3, wherein the ratio of the amount of the disintegrant present in the layer comprising quetiapine to the amount of the disintegrant present in the layer comprising escitalopram is between 2/1 and 20/1.

5. The tablet formulation according to claim 3, wherein the disintegrant is selected from the group consisting of alginic acid and alginates, ion-exchange resins, magnesium aluminum silicate, sodium dodecyl sulfate, sodium carboxymethyl cellulose, croscarmellose sodium, cross linked polyvinylpyrrolidone, carboxymethylcellulose calcium, docusate sodium, guar gum, corn starch, polacrilin potassium, poloxamer, povidone, sodium alginate, sodium glycine carbonate, sodium lauryl sulfate, sodium starch glycolate, and mixtures thereof.

6. The tablet formulation according to claim 1, wherein the ratio of the inert layer thickness to the total tablet thickness is between 1/60 and 1/4.

7. The tablet formulation according to claim 6, wherein the ratio of the inert layer thickness to the total tablet thickness is between 1/40 and 1/6.

8. The tablet formulation according to claim 1, further comprising microcrystalline cellulose.

9. The tablet formulation according to claim 8, wherein the amount of microcrystalline cellulose is 10.0 to 50.0% in the quetiapine containing layer, 60.0 to 85.0% in the escitalopram containing layer, 70.0 to 90.0% in the inert layer.

10. The tablet formulation according to claim 9, wherein the amount of microcrystalline cellulose is 20.0 to 40.0% in the quetiapine containing layer, 70.0 to 80.0% in the escitalopram containing layer, 75.0 to 85.0% in the inert layer.

11. The tablet formulation according to claim 1, further comprising one or more fillers, binders, lubricants, glidants, coloring agents, coating agents, or a mixture thereof in addition to microcrystalline cellulose.

12. The tablet formulation according to claim 11, wherein the filler is selected from the group consisting of mannitol, spray-dried mannitol; dibasic calcium phosphate dihydrate, lactose, sugars, sorbitol, lactose monohydrate; a mixture of mannitol, polyplasdone and syolid; a mixture of mannitol, crospovidone and polyvinyl acetate; isomalt, sucrose, inorganic salts such as calcium salts, and mixtures thereof.

13. The tablet formulation according to claim 11, wherein the binder is selected from the group consisting of natural gums, starch, gelatin, polyvinylpyrrolidone, polymethacrylates; proteins such as gelatin, collagen; agar, alginate, sodium alginate, pectin, starch, carboxymethyl cellulose, hydroxypropyl cellulose, hydroxypropylmethyl cellulose; synthetic polymers such as carbomer, poloxamer, polyacrylamide, polyvinyl alcohol; inorganic substances such as aluminum hydroxide, bentonite, laponite; starch mucilage, acacia mucilage, polydextrose, polyethylene oxide, and mixtures thereof.

14. The tablet formulation according to claim 11, wherein the lubricant or glidant is selected from the group consisting of sodium stearyl fumarate, magnesium stearate, polyethylene glycol, colloidal silicon dioxide, stearic acid, talk, metal stearates, boric acid, sodium chloride benzoate and acetate, sodium or magnesium lauryl sulfate, and mixtures thereof.

15. A method of preventing or treating a disease selected from the group consisting of bipolar disease, obsessive-compulsive disorder, and schizophrenia, mania, depression, dementia, panic disorders, social phobia, generalized anxiety disorder, and agitation in the humans, the method comprising administering the tablet formulation of claim 1 to a subject.
